(19) United States
(12) Patent Application Publication
(10) Pub. No.: US 2019/0284722 A1
(43) Pub. Date: Sep. 19, 2019

(54) MULTI LAYER SCAFFOLD DESIGN WITH SPACIAL ARRANGEMENT OF CELLS TO MODULATE TISSUE GROWTH

(71) Applicant: Biostage, Inc., Holliston, MA (US)

(72) Inventors: Sherif Soliman, Holliston, MA (US); Linghui Meng, Holliston, MA (US); Shunfu Hu, Holliston, MA (US)

(21) Appl. No.: 16/356,611

(22) Filed: Mar. 18, 2019

Related U.S. Application Data
(60) Provisional application No. 62/644,318, filed on Mar. 16, 2018.

(51) Int. Cl. D01D 5/00; C12N 5/0775; C12N 5/077; A61L 27/36; A61L 27/38

(52) U.S. Cl. CPC D01D 5/0015; C12N 5/0663; A61L 27/3882; A61L 27/3679; A61L 27/3834; C12N 5/0661

(57) ABSTRACT

A multilayer scaffold device that includes a luminal electrospun layer, the luminal electrospun layer configured to provide a suitable environment to induce epithelium formation on the scaffold, an exterior electrospun layer, the exterior electrospun layer located radially exterior to the luminal electrospun layer, the exterior electrospun layer configured to induce formation of non-epithelial tissue; and at least one intermediate layer interposed between the luminal electrospun layer and that exterior electrospun layer, the intermediate layer configured to organize the formation of the respective epithelial tissue and the non-epithelial tissue.

### CROSS-REFERENCE TO RELATED APPLICATION(S)

This application claims priority to and the benefit of U.S. Provisional Patent Application Ser. No. 62/644,318 filed Mar. 16, 2018, the entire disclosure of which is hereby incorporated by reference.

### TECHNICAL FIELD

The present disclosure pertains to multilayer scaffold designs. More particularly, the present disclosure pertains to multilayer scaffolds that can be employed to modulate tissue growth in tubular organs such as the esophagus.

### BACKGROUND

The esophagus is a tube connecting the pharynx with the stomach, through which food passes. In 2016, 16,910 new cases of esophageal cancer were estimated to occur, leading to about 15,690 deaths in America. In addition, birth defects as esophageal atresia or complications of gastroesophageal reflux diseases like Barrett's esophagus require surgical intervention. Esophageal cancer often requires resection of the damaged portion of the esophagus via an esophagectomy. In this procedure, diseased tissue is excised, and the stomach, jejunum, or colon is used to reconstruct the esophagus. Esophageal atresia may also require such procedures. Morbidities such as anastomotic leaks, cardiopulmonary complications, and infection result in a median survival ranging from 13 to 19 months. Tissue engineered tubular grafts present an alternative strategy, as they could replace excised esophageal tissue, and thus, restore the integrity and continuity of the esophagus with reduced complications.

The esophagus is comprised of four layers: mucosa, submucosa, muscularis propria, and adventitia. Mucosa is a non-keratinized squamous epithelium which covers the inner surface of the esophagus and its epithelium produces mucous secretions, helping the lubrication of ingested food. The submucosa also contains glands releasing important secretions for esophageal clearance and tissue resistance to acid. Motor function is insured by the muscularis propria, which is composed of striated and smooth muscle. The sequence of smooth muscle contraction and relaxation (peristalsis) propels bolus and liquids into the stomach. Thus, to fully reconstruct the structure and function of the esophagus, focus should be placed on achieving spatial organization of cells to promote the restoration of the esophageal tissue layers.

Several approaches were already considered to form a tissue-engineered esophagus. Previous studies used collagen sheets, Poly(glycolic acid) meshes, and silicon meshes. However, these studies focused on creating an epithelial layer, and lacked the multi-tissue hierarchical structure of the esophagus. Other studies aimed at creating a composite hybrid tissue by combining cultured sheets of epithelial and smooth muscle tissues, but this method was not suitable for widespread clinical use, as it carried the risk of delamination of the layers. Multilayer esophageal scaffolds were also considered, fabricated in poly(L-lactide-co-caprolactone) (PLLC) with thermally induced phase separation (TIPS) technique, or with a combination of several materials and techniques. However, those scaffolds were seeded with only one cell type, limiting the regenerative power to induce multiple tissue layers. Designing a single scaffold that can accommodate several cell populations has shown to be challenging.

Treatment of various diseases of tubular organs such as the esophagus may require resectioning of the damaged portion. The current standard of care requires the replacement of the esophagus with the stomach or the intestine. Such procedures have high rates of mortality and morbidity; therefore, the use of alternative conduits is needed.

In the past, the use of cadaver-derived tubular structures has been suggested. Also suggested is the use of materials composed of bioabsorbable material that can be integrated into the developing cellular material.

Heretofore the ability to achieve tissue regeneration and organ regrowth has been limited and difficult. It would be desirable to provide a removable structure that can be positioned so as to be oriented proximate to the anastomosis or desired target region of a tubular organ such as an esophagus and promote organized native tissue growth.

### SUMMARY

Disclosed herein are implementations of a multilayer scaffold device that includes a luminal electrospun layer, the luminal electrospun layer configured to provide a suitable environment to induce epithelium formation on the scaffold, an exterior electrospun layer, the exterior electrospun layer located radially exterior to the luminal electropsum layer, the exterior electrospun layer configured to induce formation of non-epithelial tissue; and at least one intermediate layer interposed between the luminal electrospun layer and that exterior electrospun layer, the intermediate layer configured to organize the formation of the respective epithelial tissue and the non-epithelial tissue.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is a cross-sectional depiction of a section of an embodiment of the of the multi-layer scaffold with cells seeded thereon;

FIG. 2 is perspective view of an embodiment of the multilayer scaffold as disclosed herein;

FIG. 3 is a scanning electro-micrograph (SEM) of a cross sectional view of the scaffold of FIG. 2 magnified with a scale bar at 100 μm as illustrated;

FIG. 4 is a scanning electro-micrograph (SEM) of a cross sectional view of a unilayer scaffold constructed as disclosed herein having a narrow pore configuration magnified with a scale bar at 100 μm as illustrated;

FIG. 5 is a scanning electro-micrograph (SEM) of a cross sectional view of a unilayer scaffold constructed as disclosed herein having a broad pore configuration magnified with a scale bar at 100 μm as illustrated;

FIG. 6 is a representative SEM image from the luminal aspect of the scaffold of FIG. 2 at a scale at 20 μm;

FIG. 7 is a representative SEM image from the exterior aspect of the scaffold of FIG. 2 at a scale at 20 μm;

FIG. 8 is a representative SEM image from the luminal aspect of the scaffold of FIG. 4 at a scale at 20 μm;

FIG. 9 is a representative SEM image from the exterior aspect of the scaffold of FIG. 4 at a scale at 20 μm;

FIG. 10 is a representative SEM image from the luminal aspect of the scaffold of FIG. 5 at a scale at 20 μm;

FIG. 11 is a representative SEM image from the exterior aspect of the scaffold of FIG. 5 at a scale at 20 μm;

FIG. 12 is a graphic depiction of diameter of fibers for the scaffolds of FIGS. 2, 4 and 5 with measurements from luminal aspect depicted in grey and exterior aspects depicted in white (mean±SEM, *ANOVA p<0.01);

FIG. 13 is a graphic depiction of average pore size in electrospun unilayer scaffolds using experimental and theoretical methods pore diameters derived from mercury porosimetry (grey bars) and a theoretical model (white bars) method. Mean±SEM. *ANOVA p<0.01 across the porosimetry method. *ANOVA p<0.01 between methods;

FIG. 14 is a graphic depiction of load-extension curves of electrospun scaffolds in which the plain curve corresponds to the multilayer scaffold whereas the unilayer scaffolds are associated to the dashed curves (thinly dashed for the narrow pore scaffold and largely dashed for the broad pore scaffold);

FIG. 15A are fluorescent images of viability assessment after one and seven days for SMCs on the embodiment depicted in FIG. 4;

FIG. 15B are fluorescent images of infiltration assessment after one and seven days for SMCs on the embodiment depicted in FIG. 4;

FIG. 16A are fluorescent images of viability assessment after one and seven days for SMCs on the embodiment depicted in FIG. 5;

FIG. 16B are fluorescent images of infiltration assessment after one and seven days for SMCs on the embodiment depicted in FIG. 5;

FIG. 17A are fluorescent images of viability assessment after one and seven days for MSCs on the embodiment depicted in FIG. 4;

FIG. 17B are fluorescent images of infiltration assessment after one and seven days for SMCs on the embodiment depicted in FIG. 4;

FIG. 18A are fluorescent images of viability assessment after one and seven days for SMCs on the embodiment depicted in FIG. 5;

FIG. 18B are fluorescent images of infiltration assessment after one and seven days for SMCs on the embodiment depicted in FIG. 5; and

FIG. 19 is a graphic representation of the size of space occupied by viable cells after one day and seven days.

### DETAILED DESCRIPTION

Treatment of various diseases of tubular organs such as the esophagus may require resectioning of the damaged tubular organ portion. The current standard of care requires the replacement of the esophagus with the stomach or the intestine. Such procedures have high rates of mortality and morbidity; therefore, the use of alternative conduits is needed. A tissue engineering approach that allows for the regeneration of esophageal tissues would have significant clinical application. The present disclosure presents an embodiment of a cell-seeded synthetic scaffold that can be employed to replace part of a resected tubular organ such as the esophagus of a patient and elicit tissue regrowth. Also disclosed is a multilayer scaffold device that includes a luminal electrospun layer, an exterior electrospun layer, and at least one intermediate layer that is interposed between the luminal layer and the exterior electrospun layer on which cells can be seeded such that the population of cells seeded on the luminal layer differs from the population of cells seeded on the exterior layer.

In the method and device as disclosed, various embodiments of the multilayer scaffold device as disclosed herein can be seeded with a suitable cellular material to promote the establishment and growth of cell colonies which can adhere to the respective surfaces of the multilayer scaffold. The cell-seeded scaffold as disclosed herein can replace the resected potion of the esophagus or other tubular organ. It has been found that placement of the scaffold as disclosed herein unexpectedly elicits tissue re-growth yielding a functional organ such as an esophagus. The re-growth that is induced includes two tissue layers that are believed to be significant to the ultimate function of the resulting tubular organ—an epithelium on the luminal surface of the regenerated organ and a muscle layer on the exterior surface of the regenerated organ which results in a bioengineered tubular organ such as an esophagus having both tissue layers.

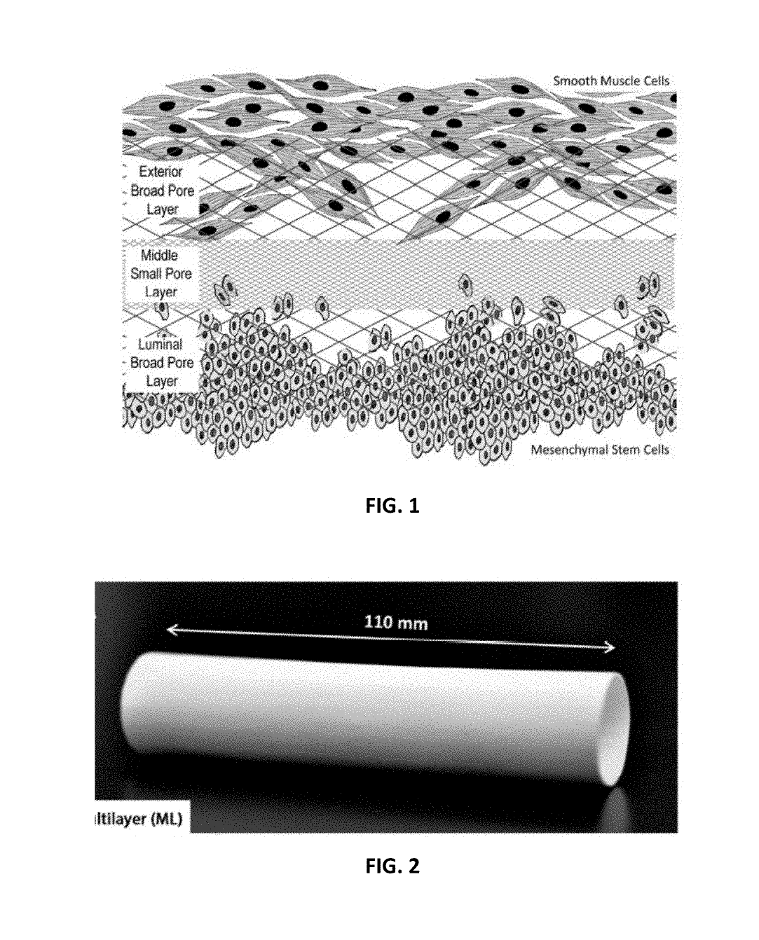

The multilayer scaffold device 10 that is disclosed herein includes luminal electrospun layer 12 and exterior electrospun layer 14. Each layer 12, 14 includes a continuous or intermittent electrospun polymeric fiber(s) 13, 15 respectively that are oriented in contacting relationship and form or define pores with broad pore sizes suffering to promote penetration and proliferation of mesenchymal stem cells (MSCs) on the respective luminal electrospun layer 12 and smooth muscle cells (SMCs) on the exterior electrospun layer 14. A non-limiting schematic diagram is depicted in FIG. 1.

The luminal electrospun layer 12 is separated from the exterior electrospun layer 14 by and intermediate layer 16 characterized by a pore size that is narrower than the pore size of the luminal electrospun layer 12 and the exterior electrospun layer 14. Where desired or required, the intermediate layer 16 can be electrospun. It is also contemplated that the respective pore sizes can be achieved via electrospinning by tuning the solution and the process parameters. Such that the resulting scaffold demonstrates production of three integrated layers with distinguishable microstructures and good mechanical integrity. Invitro validation of separated unilayer components of the multilayer scaffold can support spatial arrangement of cells needed to promote tissue regeneration.

In certain embodiments of the multilayer scaffold 10 as disclosed herein, the luminal electrospun layer 12 is composed of at least one elongated polymeric electrospun fiber 13. The at least one elongated electrospun fiber 13 in the luminal electrospun layer 12 has a first end and a second opposed to the first end and an intermediate region located between the first end and the second end. The elongated electrospun polymeric fiber is oriented such that a plurality of points of contact between different locations are defined on the intermediate region of the electrospun fiber 13. It is contemplated that the electrospun fiber 13 in the luminal electrospun layer 12 can be configured to overlay itself and define multiple layers of electrospun polymeric material.

In certain embodiments, the electrospun fiber employed in the luminal layer 12 can have a fiber diameter between 1.0 μm and 25.0 μm; between 1.0 μm and 20.0 μm; between 1.0 μm and 15.0 μm; between 1.0 μm and 10.0 μm; between 1.0 μm and 9.0 μm; between 1.0 μm and 8.0 μm; between 1.0 μm and 7.0 μm; between 1.0 μm and 6.0 μm; between 1.0 μm and 5.0 μm; between 1.0 μm and 4.0 μm; between 1.0 μm and 3.0 μm; between 1.0 μm and 2.0 μm; between 2.0 μm and 25.0 μm; between 2.0 μm and 20.0 μm; between 2.0 μm and 15.0 μm; between 2.0 μm and 10.0 μm; between 1.0 μm and 9.0 μm; between 2.0 μm and 8.0 μm; between 2.0 μm and 7.0 μm ; between 2.0 μm and 6.0 μm; between 2.0 μm and 4.0 μm; between 2.0 μm and 3.0 μm; between 3.0 μm and 20.0 μm; between 3.0 μm and 15.0 μm; between 3.0 μm and 10.0 μm; between 3.0 μm and 9.0 μm; between 3.0 μm and 8.0 μm; between 3.0 μm and 7.0 μm; between 3.0 μm and 6.0 μm; between 3.0 μm and 5.0 μm; between 3.0 μm and 4.0 μm; between 4.0 μm and 25.0 μm; between 4.0 μm and 20.0 μm; between 4.0 μm and 15.0 μm; between 4.0 μm and 10.0 μm; between 4.0 μm and 9.0 μm; between 4.0 μm and 8.0 μm; between 4.0 μm and 7.0 μm; between 4.0 μm and 6.0 μm; between 4.0 μm and 5.0 μm; between 5.0 μm and 25.0 μm; between 5.0 μm and 20.0 μm; between 5.0 μm and 15.0 μm; between 5.0 μm and 10.0 μm; between 5.0 μm and 9.0 μm; between 5.0 μm and 8.0 μm; between 5.0 μm and 7.0 μm; between 5.0 μm and 6.0 μm; between 6.0 μm and 25.0 μm; between 6.0 μm and 20.0 μm; between 6.0 μm and 15.0 μm; between 6.0 μm and 10.0 μm; between 6.0 μm and 9.0 μm; between 6.0 μm and 8.0 μm; between 6.0 μm and 7.0 μm; between 7.0 μm and 25.0 μm; between 7.0 μm and 20.0 μm; between 7.0 μm and 15.0 μm; between 7.0 μm and 10.0 μm; between 10.0 μm and 25.0 μm; between 10.0 μm and 20.0 μm; between 10.0 μm and 18.0 μm; between 10.0 μm and 17.0 μm; between 10.0 μm and 16.0 μm; between 10.0 μm and 15.0 μm; between 10.0 μm and 14.0 μm; between 10.0 μm and 13.0 μm; between 10.0 μm and 12.0 μm; between 10.0 μm and 11.0 μm; between 11.0 μm and 25.0 μm; between 11.0 μm and 20.0 μm; between 11.0 μm and 18.0 μm; between 11.0 μm and 17.0 μm; between 11.0 μm and 16.0 μm; between 11.0 μm and 15.0 μm; between 11.0 μm and 14.0 μm; between 11.0 μm and 13.0 μm; between 11.0 μm and 12.0 μm; between 12.0 μm and 25.0 μm; between 12.0 μm and 20.0 μm; between 12.0 μm and 15.0 μm; between 12.0 μm and 15.0 μm; between 12.0 μm and 14.0 μm; between 12.0 μm and 13.0 μm; between 15.0 μm and 25.0 μm; between 15.0 μm and 23.0 μm; between 15.0 μm and 22.0 μm; between 15.0 μm and 21.0 μm; between 15.0 μm and 20.0 μm; between 15.0 μm and 18.0 μm; between 15.0 μm and 17.0 μm; between 15.0 μm and 16.0 μm; between 16.0 μm and 25.0 μm; between 16.0 μm and 20.0 μm; between 16.0 μm and 18.0 μm; between 16.0 μm and 17.0 μm; between 17.0 μm and 25.0 μm; between 17.0 μm and 22.0 μm; between 17.0 μm and 20.0 μm; between 17.0 μm and 19.0 μm; between 20.0 μm and 25.0 μm; between 20.0 μm and 24.0 μm; between 20.0 μm and 23.0 μm; between 20.0 μm and 22.0 μm; between 20.0 μm and 21.0 μm.

The intermediate region of the electrospun polymeric fiber 13 employed in the luminal electrospun layer 12 can be oriented such that the there are multiple points of contact between the fiber at various locations in the intermediate region of the electrospun fiber. The polymeric fiber can be electrospun onto a suitable mandrel such that the resulting luminal electrospun layer can have between 1,000 and 1,000,000 points of contact per cubic millimeter (mm³). In certain embodiments., the number of points of contact can be between 2,000 and 1,000,000; between 5,000 and 1,000,000; between 10,000 and 1,000,000; between 50,000 and 1,000,000; between 100,000 and 1,000,000; between 500,000 and 1.000,000; between 750,000 and 1,000,000; between 1,000 and 750,000; between 2,000 and 750,000; between 5,000 and 750,000; between 10,000 and 750,000; between 50,000 and 750,000; between 100,000 and 750,000; between 500,000 and 750,000; between 1,000 and 500,000; between 2,000 and 500,000; between 5,000 and 500,000; between 10,000 and 500,000; between 50,000 and 500,000; between 100,000 and 500,000; between 250,000 and 500,000; between 1,000 and 250,000; between 2,000 and 250,000; between 5,000 and 250,000; between 5,000 and 250,000; between 10,000 and 250,000; between 50,000 and 250,000; between 100,000 and 250,000; between 1,000 and 2,000; between 2,000 and 5,000; between 2,000 and 10,000; between 5,000 and 10,000.

The luminal electrospun layer 12 in the multilayer scaffold includes an inwardly oriented luminal surface 17 and a luminal layer region 19 proximate to and immediately inward of the inwardly oriented luminal surface 17. The luminal electrospun layer 12 can have a plurality of pores such as pores 21 that are defined in the luminal electrospun layer 12. The pores 21 can have an average pores size that permits cells introduced into contact with the luminal electrospun layer to adhere to the electrospun polymeric fibers and span a portion of the pores defined therein to form cellular colonies associated with the luminal electrospun layer 12.

The luminal electrospun layer 12 in the multilayer scaffold 10 can have an average pore size great than 10.0 μm in certain embodiments. In certain embodiments, the average pore size can be between 10.0 μm and 100.0 μm; between 10.0 μm and 75.0 μm; between 10.0 μm and 50.0 μm and the like.

The pores defined by the polymeric electrospun fiber 13 in the luminal electrospun layer 12 can be a combination of pores 21 that are open at one end and through pores that communicate among themselves and function as transport conduits with the liminal layer. It is contemplated that cellular material, when introduced into contact with the inwardly oriented luminal surface 19 of the luminal layer 12, will colonize at least portions of the inwardly oriented luminal surface 19 with cellular colonies of the associated introduced cellular material.

In certain embodiments, seeded cells can also reside within the pores and interstices defined in the luminal layer 12. The seeded cells can either be introduced into the interstices or can grow into the pores and interstices through replication. Thus, in certain embodiments, a first population of cells can include a portion cells that adhere to the inwardly oriented luminal surface and a portion of cells that adhere to the luminal region proximate to and inward of the inwardly oriented surface, if desired or required. In certain embodiments, the portion of the first population of cells adhere to between 40% and 100% of the luminal surface, while in other embodiments, the portion of the luminal surface to which the first population of cells can be between 50 and 100%; 60% and 100%; 70% and 100%; 80% and 100%; 90% and 100%; 95% and 100%. In certain embodiments, the first population of cells adhering to the luminal region proximate to and inward of the inwardly oriented luminal surface constitute between 0 and 50% luminal electrospun layer 12. In other embodiments, the first population of cells adhering to the region proximate to and inward of the inwardly oriented luminal surface can make up between 1% and 40%; 1% and 30%; 1% and 20%; 1% and 10%; 1 and 5%; 1% and 4%; 1%and 3%; land 2% of that space.

In certain embodiments, the first population of cells cam be present in the luminal layer region proximate to and inward of the inwardly oriented luminal surface as a gradient with a proportion of the cells of the first population decreasing as the distance from the inwardly oriented surface of the luminal electrospun layer 12 increases.

In certain embodiments, the first population of cells that is employed to cellularize the multilayer scaffold 10 can be derived from a suitable source such as autologously derived cells. In some embodiments, the cells are progenitor or stems cells. In some embodiments, the cells are obtained from bone marrow, adipogenic tissue, esophageal tissue, or other suitable tissue. In some embodiments, the cells can be obtained from various allogenic sources, including but not limited to sources such as amniotic fluid, cord bold and the like. In some embodiments, the cells are mesenchymal stem cells (MSCs).

In certain embodiments, it is contemplated that multilayer scaffold 10 can be seeded in a manner suitable to introduce a first cell population having elevated concentration of mesenchymal stem cells (MSCs) into contact with the luminal electrospun layer 12. In certain embodiments, the percentage of mesenchymal stem cells (MSCs) in the first population of cells present on and/or in the luminal electrospun layer 12 can be greater than 40%; greater than 50%; greater than 75%.

The multilayer scaffold device 10 can also include an exterior electrospun layer 14. In the embodiment depicted, the exterior electrospun layer 14 is located radially exterior to the luminal electrospun layer 12. The exterior electrospun layer 14 is configured to induce formation of non-epithelial tissue. The exterior electrospun layer 14 can have an outwardly oriented surface 21 and a region 23 that proximate to and immediately inward of the outwardly oriented surface 21.

In certain embodiments of the multilayer scaffold 10 as disclosed herein, the exterior electrospun layer 14 is composed of at least one elongated polymeric electrospun fiber 25. The at least one elongated electrospun polymeric fiber 25 in the exterior electrospun layer 14 has a first end and a second opposed to the first end and an intermediate region located between the first end and the second end. The elongated electrospun polymeric fiber 25 is oriented such that a plurality of points of contact between different locations are defined on the intermediate region of the electrospun fiber 25. It is contemplated that the electrospun fiber 25 in the exterior electrospun layer 14 can be configured to overlay itself and define multiple layers of electrospun polymeric material

In certain embodiments, it is contemplated that the electrospun fiber employed in the exterior electrospun layer 14 can have dimensions similar to that employed in the liminal layer 12 and set forth previously.

The intermediate region of the elongated electrospun polymeric fiber 25 that is employed in the luminal electrospun layer 14 can be oriented such that the there are multiple points of contact between the fiber at various locations in the intermediate region of the electrospun fiber. The polymeric fiber can be electrospun onto a suitable mandrel such that the resulting exterior electrospun layer 14 can have between 1,000 and 1,000,000 points of contact per cubic millimeter (mm³). In certain embodiments., the number of points of contact can be between 2,000 and 1,000,000; between 5,000 and 1,000,000; between 10,000 and 1,000,000; between 50,000 and 1,000,000; between 100,000 and 1,000,000; between 500,000 and 1.000,000; between 750,000 and 1,000,000; between 1,000 and 750,000; between 2,000 and 750,000; between 5,000 and 750,000; between 10,000 and 750,000; between 50,000 and 750,000; between 100,000 and 750,000; between 500,000 and 750,000; between 1,000 and 500,000; between 2,000 and 500,000; between 5,000 and 500,000; between 10,000 and 500,000; between 50,000 and 500,000; between 100,000 and 500,000; between 250,000 and 500,000; between 1,000 and 250,000; between 2,000 and 250,000; between 5,000 and 250,000; between 5,000 and 250,000; between 10,000 and 250,000; between 50,000 and 250,000; between 100,000 and 250,000; between 1,000 and 2,000; between 2,000 and 5,000; between 2,000 and 10,000; between 5,000 and 10,000.

The exterior electrospun layer 12 in the multilayer scaffold 10 includes an exteriorly oriented luminal surface 27 and an exterior layer region 29 proximate to and immediately inward of the outwardly oriented exterior surface 27. The exterior electrospun layer 14 can have a plurality of pores such as pores 31 that are defined in the exterior electrospun layer 14. The pores 31 can have an average pores size that permits cells introduced into contact with the exterior electrospun layer 14 to adhere to the electrospun polymeric fiber 25 and span a portion of the pores 31 defined therein to form cellular colonies associated with the exterior electrospun layer 14.

The exterior electrospun layer 14 in the multilayer scaffold device 10 can have an average pore size great than 10.0 μm in certain embodiments. In certain embodiments, the average pore size can be between 10.0 μm and 100.0 μm; between 10.0 μm and 75.0 μm; between 10.0 μm and 50.0 μm and the like.

The pores defined by the polymeric electrospun fiber 25 in the exterior electrospun layer 14 can be a combination of pores 31 that are open at one end and through pores that communicate among themselves and function as transport conduits with the exterior electrospun layer. 14 It is contemplated that cellular material, when introduced into contact with the outwardly oriented surface of the exterior electrospun layer 14, will colonize at least portions of the exteriorly oriented surface with cellular colonies of the associated introduced cellular material.

In certain embodiments, seeded cells can also reside within the pores and interstices defined in the exterior layer 14. The seeded cells can either be introduced into the interstices or can grow into the pores and interstices through replication. Thus, in certain embodiments, a second population of cells can differ from the first population of cells seeded on and/or in the luminal electrospun layer 14.

In certain embodiments, the second population of cells that is employed to cellularize the exterior electrospun layer 14 of the multilayer scaffold 10 can be derived from a suitable source such as autologously derived cells. In some embodiments, the cells are progenitor or stems cells. In some embodiments, the cells are obtained from bone marrow, adipogenic tissue, esophageal tissue, or other suitable tissue. In some embodiments, the cells can be obtained from various allogenic sources, including but not limited to sources such as amniotic fluid, cord bold and the like. In some embodiments, the cells are smooth muscles cells (SMCs).

In certain embodiments, it is contemplated that exterior electrospun layer 14 of the multilayer scaffold 10 can be seeded in a manner suitable to introduce a second cell population having elevated concentration of smooth muscles cells (SMCs) into contact with the exterior electrospun layer 14. In certain embodiments, the percentage of smooth muscles cells (SMCs) in the second population of cells present on and/or in the exterior electrospun layer 14 can be greater than 40%; greater than 50%; greater than 75%.

In certain embodiments, the portion of the second population of cells adhere to between 40% and 100% of the exteriorly oriented surface, while in other embodiments, the portion of the exteriorly oriented surface to which the second population of cells adhere can be between 50 and 100%; 60% and 100%; 70% and 100%; 80% and 100%; 90% and 100%; 95% and 100%. In certain embodiments, the second population of cells adhering to the exterior electrospun layer region proximate to and inward of the inwardly oriented luminal surface constitute between 0 and 50% luminal electrospun layer 12. In other embodiments, the first population of cells adhering to the region proximate to and inward of the inwardly oriented luminal surface can make up between 1% and 40%; 1% and 30%; 1% and 20%; 1% and 10%; 1 and 5%; 1% and 4%; 1%and 3%; land 2% of that space.

In certain embodiments, it is contemplated that the cellularized material present on either the inwardly oriented lumial surface, the outwardly oriented surface or both can be configured as a cellular sheath derived from cells seeded on the multilayer scaffold during an incubation process. The cellular sheath adheres to and is in overlying relationship to the respective surface of the multilayer scaffold. It is contemplated that a major portion of the cells present in the cellular sheath will be connected to the outermost surface of the respective surface and can span pores defined therein to form a continuous or generally continuous surface.

In certain embodiments, the cellular sheath can have a thickness sufficient to provide structural integrity to the associated cellular sheath layer. In certain embodiments, the cellular sheath will be composed of a number of cells which are in contact with the respective surface of the multilayer scaffold sufficient to direct regenerating cells native to and associated with the resected tubular organ that are in contact with the sheath to produce a tissue wall that overlays the cellular sheath but does not integrate therewith. In certain embodiments, the cellular sheath can be composed of a lining that is between 1 and 100 cells thick on average. Certain embodiments can have a cell thickness between 10 and 100 cells; between 10 and 30 cells; between 20 and 30 cells, between 20 and 40 cells; between 20 and 50 cells; between 10 and 20 cells; between 30 and 50 cells; between 30 and 60 cells; between 40 and 60 cells; between 40 and 70 cells; between 70 and 90 cells.

The multilayer scaffold device 10 also includes at least one intermediate layer 16 that is interposed between the luminal electrospun layer 12 and the exterior electrospun layer 14. It has been found quite unexpectedly that the at least one intermediate layer 16 when configured as disclosed herein exerts organization on the formation of epithelial and non-epithelial tissue regenerated in situ, as from resected regions of the native tissue resident in the patient undergoing treatment.

In certain embodiments, the at least one intermediate layer 16 interposed between the luminal electrospun layer 12 and the exterior electrospun layer 14 comprises at least one elongated polymeric electrospun fiber 33. In certain embodiments, the at least one elongated polymeric electrospun fiber, the at least one elongated polymeric electrospun fiber having a fiber diameter between 1.0 μm and 25.0 μm, a first end, a second end opposed to the first end and an intermediate region located between the first end and the second end. The intermediate region of the elongated polymeric fiber 33 can be oriented such that between 2,000 and 200,000,000 points of contact between different locations on the intermediate region of the electrospun fiber 31 per square millimeter are present in the intermediate electrospun layer 16.

The polymeric fiber can be electrospun onto a suitable mandrel such that the resulting intermediate electrospun layer 16 can have between 1,000 and 1,000,000 points of contact per cubic millimeter (mm³). It is to be understood that in certain embodiments., the number of points of contact present in the intermediate electrospun layer 16 can be between 2,000 and 1,000,000; between 5,000 and 1,000,000; between 10,000 and 1,000,000; between 50,000 and 1,000,000; between 100,000 and 1,000,000; between 500,000 and 1.000,000; between 750,000 and 1,000,000; between 1,000 and 750,000; between 2,000 and 750,000; between 5,000 and 750,000; between 10,000 and 750,000; between 50,000 and 750,000; between 100,000 and 750,000; between 500,000 and 750,000; between 1,000 and 500,000; between 2,000 and 500,000; between 5,000 and 500,000; between 10,000 and 500,000; between 50,000 and 500,000; between 100,000 and 500,000; between 250,000 and 500,000; between 1,000 and 250,000; between 2,000 and 250,000; between 5,000 and 250,000; between 5,000 and 250,000; between 10,000 and 250,000; between 50,000 and 250,000; between 100,000 and 250,000; between 1,000 and 2,000; between 2,000 and 5,000; between 2,000 and 10,000; between 5,000 and 10,000 with the proviso that the number points of contact exhibited in the intermediate layer is greater than the number of point of contact in at least one of the luminal electrospun layer 12 or the exterior electrospun layer 14.

In certain embodiments, the intermediate electrospun layer 16 of the multilayer scaffold 10 as disclosed herein can include a plurality of pores 37 having an average pore size that is at least 25% smaller than the average pore size of pores present in the exterior electrospun layer 14. In certain embodiments, the intermediate electrospun layer 16 of the multilayer scaffold 10 as disclosed herein can include a plurality of pores 37 having an average pore size that is at least 25% smaller than the average pore size of pores located in the luminal electrospun layer 12.

In certain embodiments, the intermediate electrospun layer can include pores 37 that communicate between the luminal electrospun layer 12 and the exterior electrospun layer 14, the through pores 37 having an average diameter between 1.0 μm and 9.0 μm; between 1.0 μm and 8.0 μm; between 1.0 μm and 7.0 μm; between 1.0 μm and 6.0 μm between ; between 1.0 μm and 5.0 μm; between 2.0 μm and 9.0 μm; between 2.0 μm and 8.0 μm; between 2.0 μm and 7.0 μm; between 2.0 μm and 6.0 μm; between 2.0 μm and 5.0 μm; between 2.0 μm and 4.0 μm; between 2.0 μm and 3.0 μm; between 3.0 μm and 9.0 μm; between 3.0 μm and 8.0 μm; between 3.0 μm and 7.0 μm; between 3.0 μm and 6.0 μm; between 3.0 μm and 5.0 μm; between 3.0 μm and 4.0 μm; between 4.0 μm and 9.0 μm; between 4.0 μm and 8.0 μm; between 4.0 μm and 7.0 μm; 4.0 μm and 6.0 μm between ; between 4.0 μm and 5.0 μm; between 5.0 μm and 9.0 μm; between 5.0 μm and 8.0 μm; between 5.0 μm and 7.0 μm; between 5.0 μm and 6.0 μm; between 6.0 μm and 9.0 μm; between 6.0 μm and 8.0 μm; between 6.0 μm and 7.0 μm.

It has been found, quite unexpectedly, that the electrospun structure of the multilayer scaffold device as disclosed herein induces microstructures that mimic the environment of an extracellular matrix and can provide a process that permits enables direct control of certain microstructure characteristics as by the tuning of other microstructure characteristics, especially the fiber diameter.

The polymeric material employed in one or more of the layers, 12, 14, 16 can be one that is suitable for electrospinning in order to provide fabrication of desired and consistent fibers with easily tunable morphological properties. In certain embodiments, the polymeric material employed will be a polymer that includes a polycarbonate-based polyurethane polymer. In certain other embodiments. it is contemplated that the polymeric material can be composed in whole or in part of biodegradable polymers if desired or required.

It has been found, quite unexpectedly that three-dimensional structure present in the multilayer scaffold as disclosed herein provides a structure that takes into account the needed vascularization necessary to support the regenerating engineered organ. The cells seeded into the luminal electrospun layer 12 can be cells such as mesenchymal cells. It has been found that MSC's seeded into the lumen can help promoting tissue repair/regeneration process. Moreover, it has been found that cells so seeded on the luminal electrospun layer 12 can provide a suitable environment to induce epithelium formation on a scaffold, emanating from the distal native esophageal epithelial tissue. It has also been discovered that MSC's seeded on the exterior electrospun layer 14 may enhance the formation of a muscular layer enabling peristalsis.

The device 10 as disclosed herein can be configured to support two cell populations. The device as disclosed also structures to proliferation of the seeded cells in a manner that facilitates organization of cell populations in a manner similar to the organization of native tissue applications. Without being bound to any theory it is believed that the device 10 so seeded can trigger regeneration of cellularly differentiated tissue.

Without being bound to any theory, it is believed that a structure that combines broad pores in the luminal elelctropsun layer and the exterior layer that are separated by a thin narrow pore layer promotes penetration of one cell type on each side and can enable vascularization and diffusion of nutrients and oxygen will the intermediate layer possesses narrow pores of sufficient size to act as a barrier to prevent cellular translocation and/or to achieve spatial arrangement of the respect cell colonies.

In certain embodiments, it is contemplated that at least one of the luminal electrospun layer 12 or the exterior electrospun layer 14 will have an average pore size of 10 μm or greater with the intermediate layer 16 has an average pore size that is less than the pore of the respective luminal electrospun layer 12 and/or exterior electrospun layer 14. In certain embodiments, the average pore size of the intermediate layer 16 can be between 10 and 25% less than the average pore size of the respective luminal electrospun layer 12 and/or exterior electrospun layer 14. In certain embodiments, the average pore size of the intermediate electrospun layer 16 can be less than 10 μm.

In certain embodiments of the scaffold device 10 as disclosed herein will comprise at least one first population of cells 18 adhering to at least one of the exterior electrospun layer 14 or the luminal electrospun layer 12. The first cell population 18 will be composed of suitable cells. Non-limiting examples of suitable stem cell populations include mesenchymal stem cells (MSCs), smooth muscle cells (SMCs) and the like.

It is also within the purview of this disclosure that the scaffold device 10 as disclosed herein is composed of a luminal electrospun layer 12 that is positioned axially inward of an intermediate layer 16. The luminal electrospun layer 12 is an electrospun polymeric material that has an axial thickness and a plurality of pores 20 having a luminal average pore size value located on at least a portion of the axial thickness. In certain embodiments, the luminal electrospun layer 12 will include pores 20 located proximate to the luminal surface 22. It is also considered within the purview of the present disclosure for the luminal electrospun layer 12 to include pores 20 extending from the luminal surface into the axial interior therein. In certain embodiments, the pores 20 present in the luminal electrospun layer 12 will have a pore size sufficient to maintain individual cells in position. In certain embodiments, the pores 20 present in the electrospun luminal layer 12 has an average pore size greater than 10 μm. In certain embodiments, at least a portion of the individual pores 20 can be interconnected to one another in a manner to permit passage of fluids, nutrients and the like.

The intermediate layer 16 is positioned axially outward from the electrospun luminal layer 12. In the embodiment as illustrated in FIGS. 1 and 2, the intermediate layer 16 is contiguously connected to the electrospun luminal layer 12 at a location distal to the luminal surface 22.

In certain embodiments, the intermediate layer 16 is an electrospun polymeric material that can have a plurality of pores 24 having an average pores sizes that is less than the average pore size value of the pores 20 present in the electrospun luminal layer 12. In certain embodiments, the average pore size value of the pores 24 present in the intermediate layer 16 are sufficient to permit transit of therethrough but to impeded transit of individual cells, for example SMCs and MSCs, therethrough. In certain embodiments, the pores present in the intermediate layer have an average pore size that is less than 10 μm.

The exterior electrospun layer 14 is positioned axially outward from the intermediate layer 16. In the embodiment as disclosed herein, the exterior electrospun layer 14 is contiguously connected to the intermediate layer 16 at a location distal to the luminal electrospun layer 12. The exterior electrospun layer 14 has an exterior surface 26 that is opposed to the position of the intermediate electrospun layer 16.

The exterior electrospun layer 14 is an electrospun polymeric material that has an axial thickness and a plurality of pores 28 having a luminal average pore size value located on at least a portion of the axial thickness. In certain embodiments, the electrospun exterior layer 14 will include pores 28 located proximate to the exterior surface 26. It is also considered within the purview of the present disclosure for the exterior electrospun layer 14 to include pores 28 extending from the exterior surface 26 into the interior therein. In certain embodiments, the pores 28 present in the exterior electrospun layer 14 will have a pore size sufficient to maintain individual cells in position. In certain embodiments, the pores 28 present in the exterior electrospun layer 14 has an average pore size greater than 10 μm. In certain embodiments, at least a portion of the individual pores 28 can be interconnected to one another in a manner to permit passage of fluids, nutrients and the like.

In the embodiment as depicted in FIGS. 1 and 2, the scaffold 10 at least has one first population of cells 18 adhering to the luminal electrospun layer 12. The first cell population 18 will be composed of suitable cells. Non-limiting examples of suitable stem cell populations include mesenchymal stem cells (MSCs), smooth muscle cells (SMCs) and the like. The scaffold 10 also includes at least one second population of cells 30 adhering to at exterior electrospun layer 14. The second cell population 30 will be composed of suitable cells that differ from the first cell population 18. Non-limiting examples of the second cell population 30 include mesenchymal stem cells (MSCs), smooth muscle cells (SMCs) and the like. In the embodiment as illustrated in FIGS. 1 and 2, the first population of cells 18 is composed of MSCs and the second population of cells is composed of SMCs.

Without being bound to any theory, it is believed that the multilayer scaffold 10 as disclosed herein supports both MSCs on the luminal aspect and SMCs on the exterior aspect, with a small pore layer in the middle to separate the two cell populations in a manner that recreates the spatial arrangement present in organs such as a patient's native esophagus needed for a functional organ, with the MSCs promoting angiogenesis and SMCs providing the muscle layer needed for peristalsis. It is believed that an epithelium could grow from the remaining distal tissue such as epithelial tissue to cover the lumen of the scaffold 10. After being seeded with autologous patient's cells, this scaffold could serve as an alternative treatment for esophageal diseases, replacing the damaged part of the esophagus and enabling its regeneration.

Thus, the scaffold 10 as illustrated in FIGS. 1 and 2 can be employed to replace the resected part of the esophagus and elicit tissue re-growth inducing at least two tissue layers: an epithelium on the luminal surface and a muscle layer on the exterior surface. In the process as disclosed, the scaffold 10 includes Luminal and exterior layers were electrospun with broad pore size to promote penetration and proliferation of mesenchymal stem cells (MSCs) on the lumen and smooth muscle cells (SMCs) on the external. The two layers are separated by a thin layer with substantially narrower pore size intended to act as a barrier for the two cell types. This multilayer scaffold design is achieved electrospinning by tuning the solution and the process parameters. Analysis of the scaffold demonstrated that this tuning enabled the production of three integrated layers with distinguishable microstructures and good mechanical integrity.

Also disclosed are various embodiments of a method of regenerating a tubular organ such as a gastrointestinal organ. In certain embodiments, the method includes the step of resecting that comprises resecting a portion of a tubular organ in a subject. The organ to be resected can be a tubular organ of the gastrointestinal tract that has been damaged or compromised by disease, injury, trauma or congenital conditions. In certain embodiments, non-limiting examples of suitable organs include one of the esophagus, rectum and the like. In certain embodiments, suitable organs include at least one of the esophagus, small intestines, colon, rectum.

The resection can be achieved by any suitable surgical procedure and produce a resected organ portion that remains connected to the gastrointestinal tract and remains in the subject after resection. The resection operation can yield suitable resection edges in certain embodiments.

After resection is completed, the multilayer synthetic scaffold as disclosed herein is implanted at the site of the resection. In certain embodiments, implantation can include the step of connecting the respective ends of the resected organ remaining in the subject to respective opposed ends of the synthetic scaffold such that the synthetic scaffold and the resected organ can achieve a suitable junction between the respective members. This can be achieved by one or more of sutures, bioorganic tissue glue, etc.

Various embodiments of the synthetic scaffold have been discussed and can be employed and utilized in the method disclosed herein. In certain embodiments, the synthetic scaffold will include a first end and a second end opposed to the first end, an outer polymeric surface positioned between the first end and the second end and a cellularized sheath layer overlying at least a portion of the outer polymeric surface. In certain embodiments, the implantation step can be one that brings at least a portion of the cellular material such as a cellularized sheath layer into proximate contact with to at least one of the resection edges of the resected organ portion.

In certain embodiments, the method as disclosed herein also includes the step of maintaining the synthetic scaffold at the resection site for a period of time sufficient to achieve guided tissue growth along the synthetic scaffold. In certain embodiments, the guided tissue growth is derived from and is in contact with the tissue present in the resected organ portion remaining in the subject. In certain embodiments, the guided tissue growth will be contiguous with the associated regions of the resected organ. In certain embodiments, the guided tissue growth will exhibit differentiated tissue. In certain embodiments, the guided tissue growth will parallel the outer surface of the cellularized sheath layer at a position outward thereof. In certain embodiments, the guided tissue growth is derived from and is in contact with the tissue present in the resected organ portion remaining in the subject and will be contiguous with the associated regions of the resected organ. The guided tissue growth will exhibit differentiated tissue growth and can be parallel the outer surface of the cellularized sheath layer at a position outward thereof.

After the guided tissue growth has been achieved, the process as disclosed herein can include step of removing the synthetic scaffold. In certain embodiments, the removing step occurs in a manner such that the guided tissue growth remains in the contact with the resected portion of the organ remaining in the subject. In certain embodiments, the removal process can include intrascopically removing the synthetic scaffold from the interior of the guided tissue growth.

In certain embodiments, the synthetic scaffold can be constructed in whole or in part from bioabsorbable polymeric material. In such situations, the method as disclosed herein can include the step of maintaining contact between the synthetic scaffold and the resection edge for an interval sufficient to achieve guided tissue growth along the synthetic scaffold such that at least a portion of the synthetic scaffold is absorbed at the site of resection within a period of time sufficient to achieve guided tissue growth along the synthetic scaffold. In certain embodiments where the scaffold is composed entirely of bioabsorable material, the scaffold will be configured to maintain structural integrity during guided tissue growth. In certain embodiments, where the synthetic scaffold is composed of bioabsorbable material in selected regions, it is contemplated that the remainder of the scaffold can be removed by suitable procedures after the guided tissue growth has been achieved.

Guided tissue growth can be monitored by suitable means. In certain embodiments, tissue growth can be monitored endoscopically.

Without being bound to any theory, it is believed that implanting a synthetic multilayer scaffold such as those as variously disclosed herein, particularly one seeded with cellular material as disclosed herein , promotes growth, regeneration and differentiation of the subject tissue in contact with or proximate to the location of the implanted synthetic multilayer scaffold. The growing regenerating tissue is guided by the synthetic scaffold structure and by signaling emanating from the extracellular matrix-like structure and associated cellular material to produce a tubular cellular body that is integrally connected to the resected ends of the remaining tubular organ and can be outwardly flaring to encapsulate the synthetic scaffold and associated cellular layer. It is believed that the scaffold and associated cellular material may promote or stimulate regenerative growth of the resected tissue while minimizing tissue rejection responses. It is also believed that the presence of the cellular material can reduce or minimize penetration of the regenerated tissue into the sheath layer during growth and differentiation. In certain embodiments, tissue generation proceeds from the respective ends toward the middle.

To further illustrate the present disclosure, the following non-limiting examples are presented.

### EXAMPLE I

Scaffold fabrication—Three types of scaffolds were electrospun: a) a multilayer (ML) scaffold with two broad pore layers separated by narrow pore layer as defined herein, b)a unilayer scaffold with narrow pores (NP), and c) a unilayer scaffold with broad pores (BP) (Instrument: IME Technologies, Geldrop, Netherlands). Droplets of polycarbonate-based polyurethane (PCU) in hexafluoroisopropanol (HFIP, DuPont, Wilmington, USA) (8% w/v for the (NP); 15% w/v for the (BP)) were charged (NP: 16 kV; BP: 14 kV) at the tip of a blunt needle (NP: 22 G; BP: 18 G, New England Small Tube, Litchfield, NH) and dispensed at constant flow (NP: 3 mL/h; BP: 15 mL/h) onto a grounded, rotating aluminum mandrel with 22 mm diameter (NP: 500 rpm; BP: 200 rpm) placed 27 cm from the needle. A total of either 14 mL or 8 mL of the polymer solution were used for each NP or BP scaffold, respectively. For the multilayer scaffold, 4 mL were electrospun for each BP layer and 8 mL for the NP layer. The time between spinning processes was less than thirty minutes. All scaffolds were electrospun at 23° C. and 30% humidity.

Post-treatment of scaffolds—The three electrospun scaffolds were each dried in a vacuum oven at 60° C. for 20 hours to remove residual solvent. Dry scaffolds were treated using a low-pressure oxygen plasma system (Tetra 150-LF-PD-D, Diener, Ebhausen, Germany) to enhance wettability. Plasma treated scaffolds were sterilized by gamma irradiation (25-30 kGy, STERIS AST, Northborough, Mass.).

Scaffold morphology—Samples from all scaffolds were coated with platinum and palladium for 70 seconds (108 Auto Sputter Coater, Cressington, Ted Pella Inc, Redding, Calif.). Samples were imaged using a scanning electron microscope (SEMEVO MA-10, Carl Zeiss, Thornwood, N.Y.) with a beam acceleration of 10 kV.

Scaffold fiber diameter—Fiber diameters of each of the samples were measured from SEM images using analysis software (FibraQuant 1.3.153, NanoScaffold Technologies, Chapel Hill, N.C.). The software automatically measures fiber diameter distribution, fiber orientation, and fiber area coverage from SEM images of fibrous and membrane materials. The software performs hundreds of measurements, which are displayed on the image, while their corresponding values are shown on an interactive table and histogram. Besides the fully automated mode, the analysis can be enhanced with versatile semi-manual and manual editing tools for complete control over the extent and the accuracy of the analysis. At least 250 measurements were recorded on each scaffold type using top view SEM images of 2000×.

Scaffold pore size—Pore size was estimated for unilayer scaffolds experimentally using mercury porosimetry and theoretically using a mathematical model. For the mercury porosimetry method, three samples from each scaffold (two of 20×15 mm and one of 20×10 mm) were weighed and placed in the sample penetrometer of the mercury porosimeter (AutoPore IV 9500, Micromeritics, Norcross, Ga.). The sample penetrometer (initially 0.2 psia) was filled with mercury to a pressure of 30 psia for the BP samples and 20,000 psia for the NP samples. These pressures detected the diameters of pores between 6-850 μm (low pressure) and 0.036-850 μm (high pressure). All samples were analyzed at low pressure; the NP samples were additionally measured at high pressure. The pore size was also estimated through approximate statistical model.

Mechanical strength testing—A 5 mm×20 mm sample from each scaffold type was stretched on an electromechanical load frame (5943 Apparatus, 1 kN load cell, Instron, Norwood, Mass.) using a 0.2 mm/s deformation speed. Tensile testing was conducted under ASTM D638 standard.

Cell seeding and incubation—Porcine adipose-derived mesenchymal stem cells (La Francesca S et al. Esophageal regeneration with a cell-seeded tissue engineered graft. Nat Biomed Eng 2017) and human esophageal smooth muscle cells (ESMCs) (Sciencell, Carlsbad, Calif.) were seeded onto scaffolds and cultured at 37° C. and 5% CO₂. In order to evaluate the interaction between the different cell types with the different electrospun layers separately, only unilayer NP and BP scaffolds were seeded. Four 2 cm×2 cm sections were obtained from each unilayer scaffold type. Two sections were seeded with MSCs on the luminal aspect and two with ESMCs on the exterior aspect. Samples of scaffold were placed into non-tissue culture treated 6-well plates and seeded with a58 μL it drop containing 250,000 cells in complete culture medium (MSCs StemXVivo, R&D Systems, Minneapolis, Minn.; SMCM, Sciencell). One of each scaffold and cell type was analyzed after 1 day and 7 days of incubation.

Cell attachment—To examine the extent of cell detachment from the scaffold, conditioned media was collected, after one day in culture, from each well and cell counts were performed using trypan blue exclusion. The collected media was centrifuged (Sorval ST 40, Thermo Scientific) at 1000 rpm and the supernatant was aspirated. The pellet was re-suspended in 0.5 mL of phosphate buffered saline. 10□1 of the suspension was mixed equally with trypan blue and loaded into a counting chamber slide for counting and viability (Countess, ThermoFisher Scientific, Waltham, Mass.).

Cell viability—Seeded scaffold sections were washed twice with phosphate buffered saline (PBS) and stained for 5 minutes in the dark with calcein AM and ethidium bromide (Live/Dead kit, ThermoFisher Scientific). After washing with PBS, the punch biopsies were imaged using an epifluorescent microscope equipped with filters to detect Green Fluorescent Protein and Texas Red fluorophores (cellSens and BX63F, Olympus, Center Valley, Pa.). Each punch was scored 0-4, according to coverage of viable cells: each image containing the complete area of cells was divided into quarters and each quarter was graded (0 or 1). A cumulative score of 0/4 indicated that the entire surface area was dead (all red), 4/4 indicated that the entire punch was viable (all green) and 2/4 indicated that half the punch was dead (red) and half was alive (green).

Radial cell translocation—By measuring images of calcein AM from days 1 and 7, radial translocation of the cells was determined. The diameter of the area stained by calcein AM for each punch was measured at four points (angles separated by 45°). The 4 diameters were averaged for each punch and the difference in values was calculated between day 1 and day 7.

Deep-layer cell translocation—Seeded scaffold sections previously stained with calcein AM were fixed in 4% paraformaldehyde, washed with phosphate buffered saline (PBS). Fixed pieces were bisected and imbedded in optimal cutting temperature (OCT) medium and frozen at −80° C.20 μm thick cryosections were cut (Cryostat, Cryostar, ThermoFisher Scientific) and mounted onto charged microscope slides (Superfrost plus, FisherScientific). Cryosections were permeabilized with 0.1% Triton-X100 in PBS (Sigma-Aldrich, St. Louis, Mo.), stained in ethidium bromide, and imaged 10× under the epifluorescent microscope (cellSens and BX63F, Olympus, Center Valley, Pa.). Each sample was scored 0-4 to assess the depth of cell translocation: 0 if cells did not attach to the scaffold, 1 if the cells were observed at the surface of the scaffold, 2 if cell translocation was observed through the superficial half of the scaffold, 3 if three quarters of the scaffold depth contained cells and 4 if cells were detected throughout the thickness of the scaffold.

Statistical analysis—Statistical analysis was performed on fiber diameters and on cell migration with MATLAB (R2016b, The MathWorks, Mass.). For fiber diameters, pore diameters and porosity, a One-Way ANOVA was applied, followed by pairwise comparison testing if the ANOVA results showed significant difference between groups (p<0.01). Bonferroni's correction was applied to counter the effects of multiple comparisons. Cell migration of each group (n=2) was assessed by a t-test applied to the viable cell area of Day 1 and Day 7. Differences were considered statistically significant when the p value was <0.01.

### EXAMPLE II

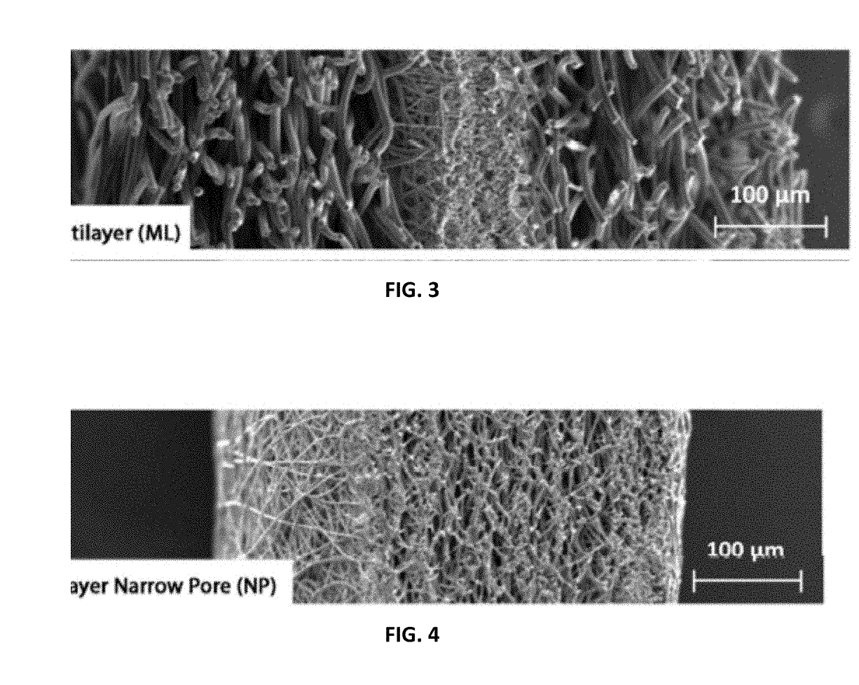

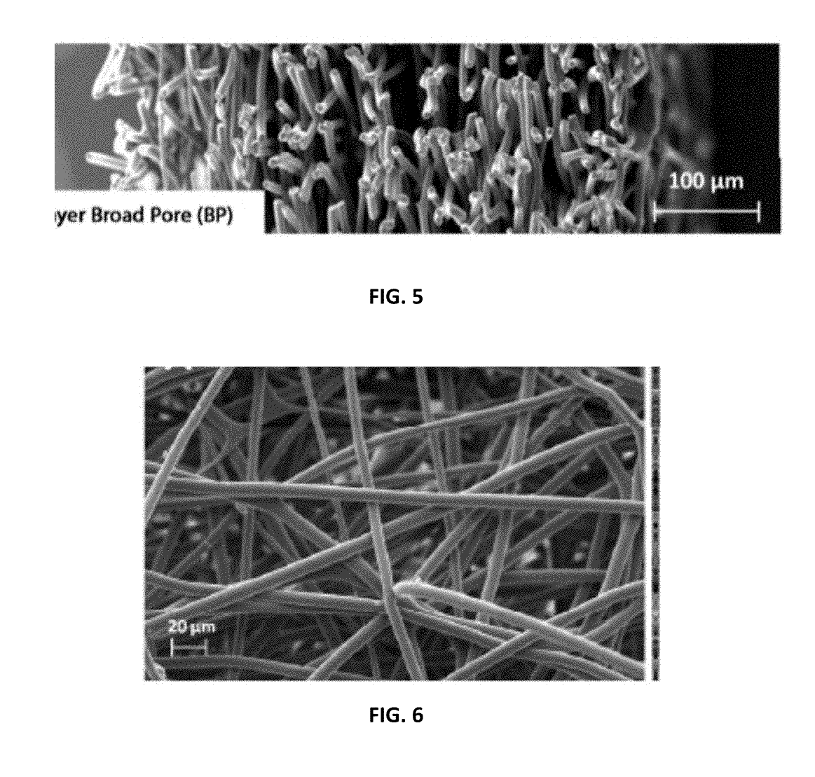

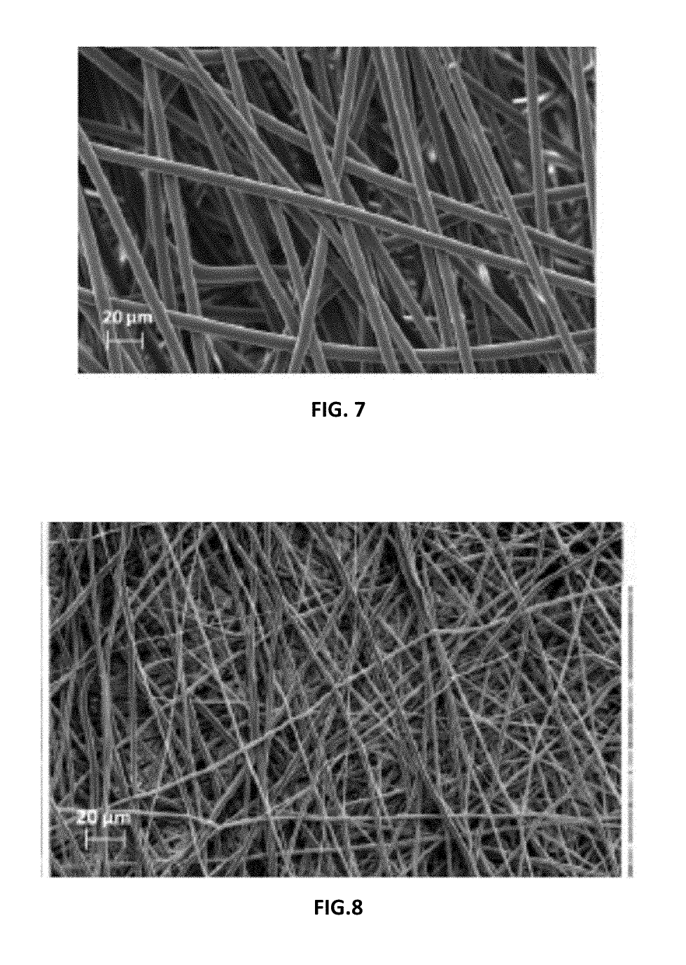

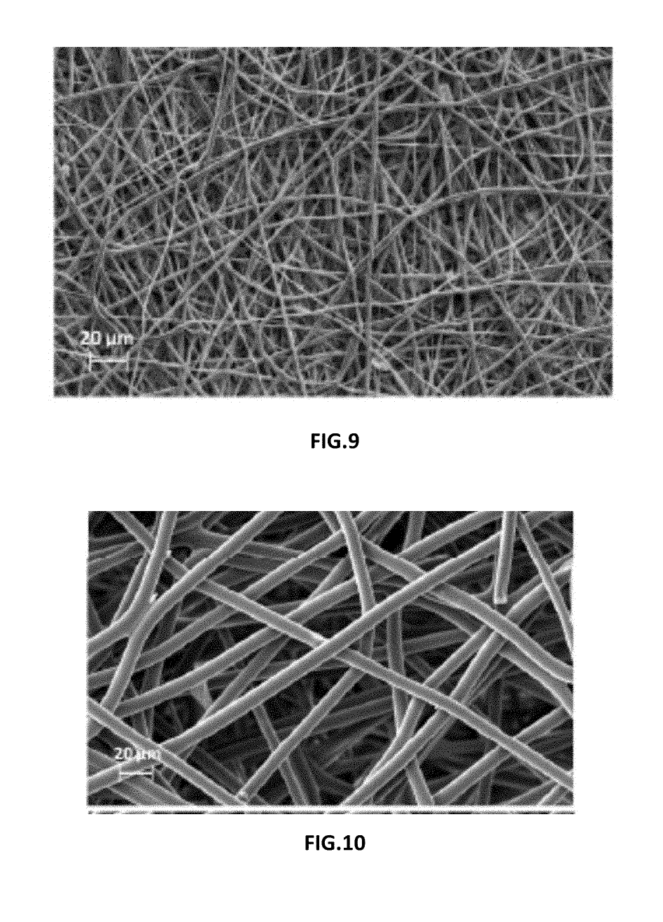

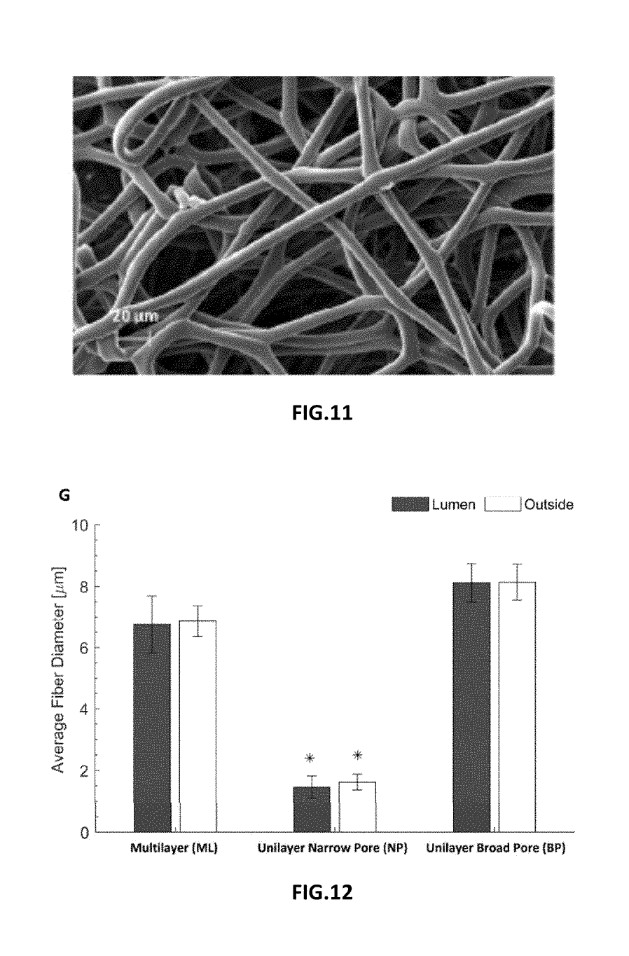

Scaffold characteristics The scaffolds produced in Example I were created as hollow cylinders, 110 mm long and 22 mm diameter as illustrated in FIG. 2. In cross-section, the fibers comprising each layer were discernable (FIGS. 2, 3 and 4). The structure of the luminal and exterior aspects of each type of scaffold were homogeneous (FIGS. 5, 6, 7, 8, 9, 10, 11). The fibers were smooth and randomly oriented for all scaffolds and no beads were observed. The fiber diameter on the luminal and exterior aspects of each scaffold type were identical (1.5 ±1.2 μm luminal and 1.6±1.2 μm exterior of narrow pore scaffold, 8.1±0.7 μm luminal and 8.1±0.4 μm exterior of BP scaffold, ANOVA *p<0.01, FIG. 12).

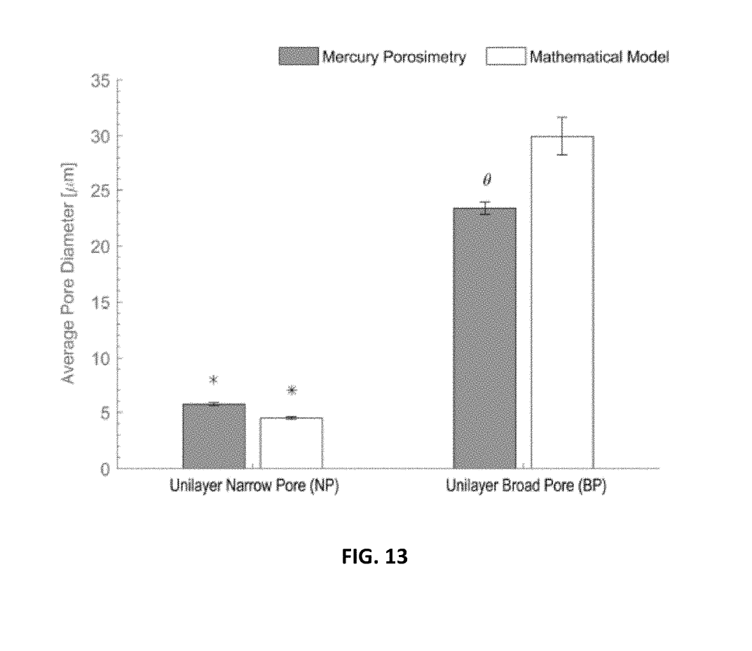

The average pore diameter for each scaffold was measured by both mercury porosimetry and a mathematical model (FIG. 13). Mercury porosimetry demonstrated that the scaffolds constructed from small fiber diameter had narrower pores than scaffolds constructed from large fiber diameter (5.7±0.3 μm and 23.3±1.0 μm for narrow pore and broad pore scaffolds respectively, ANOVA p<0.01). Similarly, the mathematical model estimated the diameter of pores to track with fiber diameter (4.5±0.2 μm and 30.0±3.3 μm for narrow pore and broad pore scaffolds respectively, ANOVA p<0.01). Between the two methods for estimating pore size, measurements of narrow pore scaffolds were concordant but the estimated diameter of the broad pore scaffolds was different between the experimental and theoretical methods (ANOVA, p<0.01).

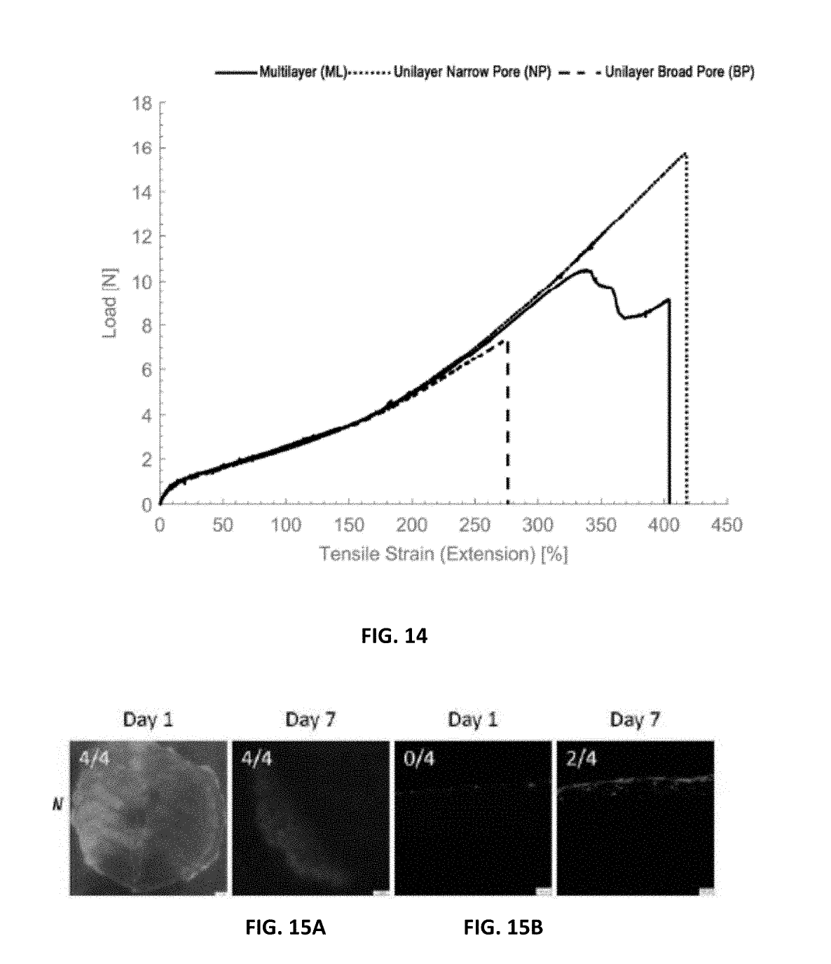

The maximum load of each scaffold type was determined and is depicted in FIG. 14. The three scaffold types had the same load until an extension of 200% was applied. The broad pore scaffold had a load increasing slower than the other scaffolds, resulting in 276% of extension with a maximum load of 7.37 N. The narrow pore scaffold had a maximum extension and load about twice as large as the broad pore scaffold (418% and 15.7 N). The multilayer scaffold broke in three times, corresponding to the three-layer delamination. First, the two exterior broad pore layers shredded one after the other (see the first two irregularities on the multilayer scaffold curve of FIG. 13), causing the load to decrease. This was followed by re-increasing the load on the remaining intact narrow pore layer. Finally, the narrow pore scaffold broke at 404% of extension. The maximum load and extension supported by the multilayer scaffold (before delamination occurred) were respectively of 10.5 N and 330%.

### EXAMPLE III

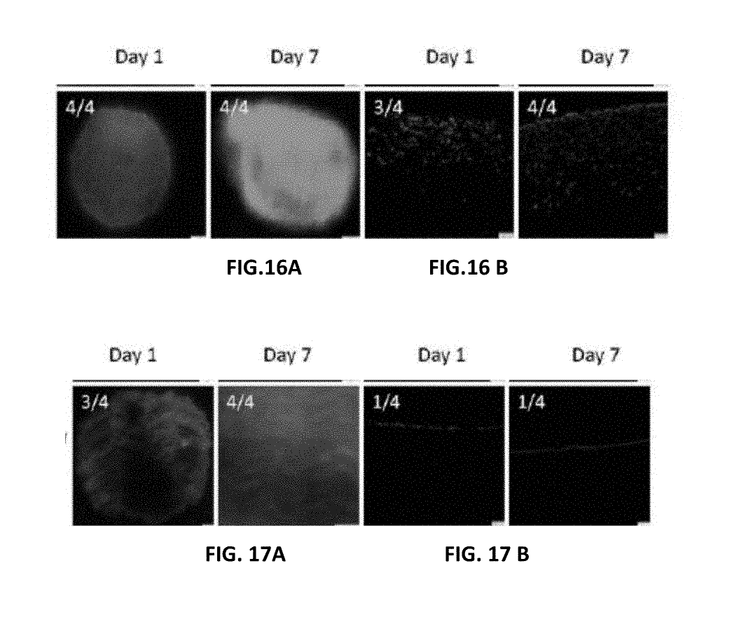

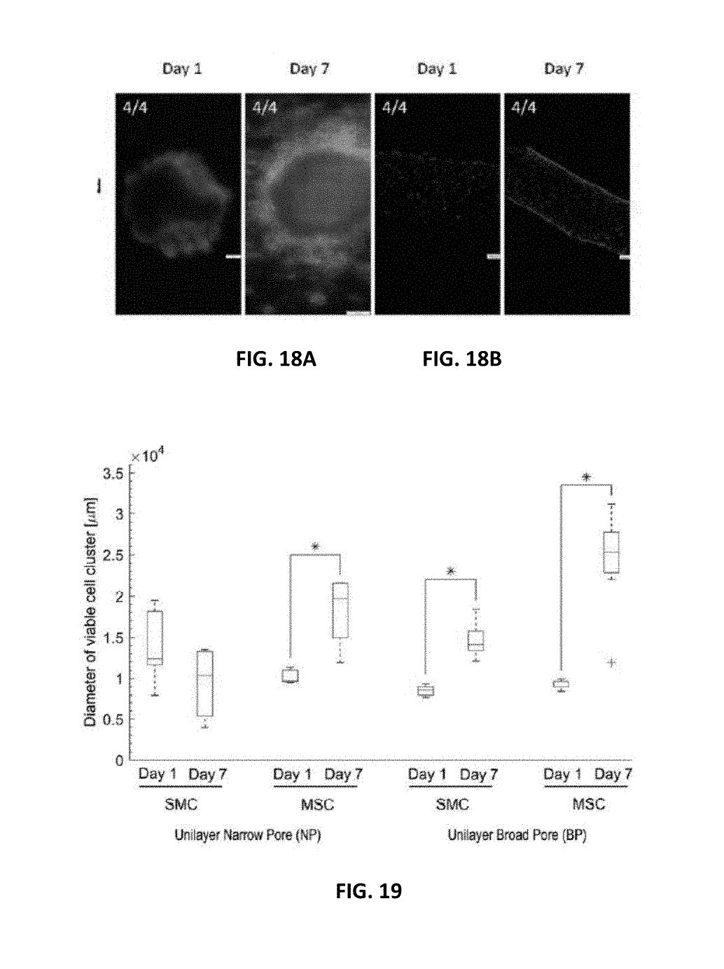

In order to determine cell attachment and viability, the effects of narrow pore and broad pore unilayer scaffolds as prepared in Example II on two cell types: mesenchymal stem cells (MSC) and smooth muscle cells (SMC) The cell types were applied to either the luminal or exterior aspect of biopsies from each unilayer scaffold. Live cell imaging of the scaffold biopsies after either 1 or 7 days in culture revealed viable MSCs and SMCs (green fluorescent, Calcine AM) with few dead cells (red fluorescent, ethidium bromide) as illustrated in FIGS. 15, 16, 17 and 18. The diameters of the population of adherent cells after 1 or 7 days in culture revealed significant SMC migration only on scaffold sections containing broad pores whereas MSCs significantly migrated on both surfaces (t-test p<0.01, FIG. 19).

Cell migration on the narrow pore scaffolds was spotty compared to a more uniformly migration on the broad pore scaffolds. The t-test result indicated a greater migration on the broad pore scaffold than on the narrow pore scaffold (slightly significant, p<0.05). Scores on four are indicated on the top-left corner of the corresponding images.

### EXAMPLE IV

Unilayer scaffold sections carrying MSCs or SMCs were fixed and sectioned to assess cell translocation into the scaffold from the surface. Both MSCs and SMCs were visible on the luminal or exterior surface of the biopsies from narrow pore scaffold after 1 day in culture. Similarly, after 7 days in culture, both MSCs and SMCs were only observed on the most superficial of the narrow pore scaffold. In contrast, both MSCs and SMCs applied to the broad pore scaffold were observed throughout the depth of the scaffold after 1 and 7 days in culture. Semi-quantitative scoring revealed that the broad pore scaffold permitted both cell types to reach deeper fibers layers within the depth of the scaffold. Scores on four are indicated on the top-left corner of the corresponding images (see FIGS. 15-18).

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A multilayer scaffold device comprising:
a luminal electrospun layer, the luminal electrospun layer configured to provide a suitable environment to induce epithelium formation on the scaffold;
an exterior electrospun layer, the exterior electrospun layer located radially exterior to the luminal electropsum layer, the exterior electrospun layer configured to induce formation of non-epithelial tissue; and
at least one intermediate layer interposed between the luminal electrospun layer and the exterior electrospun layer, the intermediate layer configured to organize the formation of the respective epithelial tissue and the non-epithelial tissue.

2. The multilayer scaffold of claim 1 wherein the luminal electrospun layer comprises at least one elongated polymeric electrospun fiber, the at least one elongated polymeric electrospun fiber having a fiber diameter between 1.0 μm and 25.0 μm, a first end, a second end opposed to the first end and an intermediate region located between the first end and the second end, wherein the intermediate region is oriented such that between 1,000 and 100,000,000 points of contact between different locations are defined on the intermediate region per square millimeter are present in the luminal electrospun layer.

3. The multilayer scaffold of claim 1 wherein the intermediate region of the at least one polymeric elongated electrospun fiber of the luminal electrospun layer has multiple points of contact per cubic millimeter and defines a plurality of pores in the luminal electrospun layer, the pores have an average pore size greater than 10.0 μm.

4. The multilayer scaffold of claim 3 wherein at least a portion pf the pores present in the luminal electrospun layer are through pores within the luminal layer.

5. The multilayer scaffold of claim 4 wherein the pores present in the luminal layer have an average pore size between 10.0 μm and 1000.0 μm.

6. The multilayer scaffold of claim 1 wherein the exterior electrospun layer comprises at least one elongated polymeric electrospun fiber, the at least one elongated polymeric electrospun fiber having a fiber diameter between 1.0 μm and 25.0 μm, a first end, a second end opposed to the first end and an intermediate region located between the first end and the second end, wherein the intermediate region is oriented such that between 1,000 and 100,000,000 points of contact between different locations are defined on the intermediate region per square millimeter are present in the exterior electrospun layer

7. The multilayer scaffold of claim 1 wherein the intermediate region of the at least one polymeric elongated electrospun fiber of the exterior electrospun layer has multiple points of contact per cubic millimeter and defines a plurality of pores in the exterior electrospun layer, the pores have an average pore size greater than 10.0 μm

8. The multilayer scaffold of claim 7 wherein the pores present in the exterior electrospun layer have an average pore size between 10.0 μm and 1000.0 μm

9. The multilayer scaffold of claim 7 wherein the at least one intermediate layer interposed between the luminal electrospun layer and the exterior electrospun layer comprises at least one elongated polymeric electrospun fiber, the at least one elongated polymeric electrospun fiber having a fiber diameter between 1.0 μm and 25.0 μm, a first end, a second end opposed to the first end and an intermediate region located between the first end and the second end, wherein the intermediate region of the elongated polymeric fiber is oriented such that between 2,000 and 200,000,000 points of contact between different locations are defined on the intermediate region of the electrospun fiber per square millimeter are present in the intermediate electrospun layer and wherein the at least one polymeric elongated electrospun fiber of the intermediate electrospun layer has multiple points of contact per cubic millimeter and defines a plurality of pores in the intermediate electrospun layer, the pores have an average pore size that is at least 25% less than the pore size of pores defined in the exterior electrospun layer.

10. The multilayer scaffold device of claim 7 wherein intermediate electrospun region has a plurality of pores communicating between the luminal electrospun layer and the exterior electrospun layer, the pores present in the intermediate electrospun layer have an average pore size less than 10 μm.

11. A multilayer scaffold device comprising:
a luminal electrospun layer, the luminal electrospun layer having an inwardly oriented luminal surface and a luminal layer region proximate to and inward of the inwardly oriented luminal surface;
an exterior electrospun layer, the exterior electrospun layer located radially exterior to the luminal electropsum layer, the exterior layer having an outwardly oriented surface and an exterior layer region proximate to and immediately inward relative to the outwardly oriented surface;
at least one intermediate electrospun layer interposed between the luminal electrospun layer and the exterior electrospun layer;
a first population of cells, where in a portion of the first population of cells adheres to the inwardly oriented luminal surface and an additional portion adheres to the luminal layer region proximate to and inward of the inwardly oriented luminal surface; and
a second population of cells, the second populations of cells adhering to the outwardly oriented surface of the exterior electrospun layer.

12. The multilayer scaffold device of claim 11 wherein the portion of the luminal electrospun layer in contact with the first population of cells is between 50% and 100% of the luminal electrospun layer.

13. The multilayer scaffold device of claim 12 wherein the first population of cells comprises mesenchymal stem cells (MSCs), where the mesenchymal stem cells (MSCs), are present in a percentage greater than 40% of the total cells in the first cell population.

14. The multilayer scaffold device of claim 11 wherein the portion of the exterior electrospun layer in contact with the second population of cells is between 50% and 100% of the exterior electrospun layer.

15. The multilayer scaffold device of claim 14 wherein the second population of cells comprises smooth (SMCs), where the smooth muscle cells (SMCs), are present in a percentage greater than 40% of the total cells in the second cell population.

16. A method for regenerating a tubular organ, the method comprising the steps of:
resecting a portion of a tubular organ in a subject, the resection step producing a resected organ portion, the resected organ portion remaining in the subject;
implanting the multilayer of claim 11 at the site of resection;
maintaining the synthetic scaffold at the resection site for a period of time sufficient to achieve guided tissue growth along the synthetic scaffold, the guided tissue growth derived from and in contact with the tissue present in the resected organ portion remaining in the subject; and
after achieving guided tissue growth, removing the synthetic scaffold from the implantation site, the removing step occurring in a manner such that the guided tissue growth remains in the contact with the resected portion of the tubular organ remaining in the subject.

17. The method of claim 16 where the removal is achieved endoscopically.
